(19) United States
(12) Patent Application Publication
(10) Pub. No.: US 2016/0271226 A1
(43) Pub. Date: Sep. 22, 2016

(54) PHARMACEUTICAL COMPOSITION

(71) Applicant: WOCKHARDT LIMITED, Aurangabad O (IN)

(72) Inventors: Maharaj K. SAHIB, Aurangabad 3 (IN); Jeetendra kashinath AMBULGE, Aurangabad 431003 (IN)

(21) Appl. No.: 15/021,693

(22) PCT Filed: Sep. 29, 2014
(86) PCT No.: PCT/IB2014/064922

§ 371 Date: Mar. 12, 2016

(30) Foreign Application Priority Data
Sep. 30, 2013 (IN) 3124/MUM/2013

(51) Int. Cl. A61K 38/28

(52) U.S. Cl. CPC A61K 38/28

(57) ABSTRACT

The present invention discloses a composition comprising readily dissociable molecular aggregates formed by combining an insulin, insulin analogue, derivative or metabolite having isoelectric point between 5.8 to 8.5 in combination with one or more an insulin, insulin analogue, derivative or metabolite having isoelectric point between 4.0 to 5.7.

### FIELD OF THE INVENTION

The present invention provides a pharmaceutical composition comprising readily dissociable molecular aggregates formed by combining an insulin, insulin analogue, derivative or metabolite having isoelectric point between 5.8 to 8.5 in combination with one or more an insulin, insulin analogue, derivative or metabolite having isoelectric point between 4.0 to 5.7 optionally along with one or more excipients, wherein the said molecular aggregates have an average particle size between about 5 μm to about 20 μm. The invention also provides process for preparation of the said biphasic formulation and its use in treatment of diabetes disorders.

### BACKGROUND OF THE INVENTION

Glucose is a simple sugar used by all the cells of the body to produce energy and support life. Humans need a minimum level of glucose in their blood at all times to stay alive. The primary manner in which the body produces blood glucose is through the digestion of food. When a person is not getting sufficient glucose from food digestion, glucose is produced from stores in the tissue and released by the liver. The body's glucose levels are primarily regulated by insulin. Insulin is a peptide hormone that is naturally secreted by the pancreas. Insulin helps glucose enter the body's cells to provide a vital source of energy.

Diabetes is a disease characterized by abnormally high levels of blood glucose and inadequate levels of insulin. Diabetes is a general term for disorders in man having excessive urine excretion as in diabetes mellitus and diabetes insipidus. Diabetes mellitus (DM) is a major chronic illness found in humans with many consequences. Some complications arising from long-standing diabetes are blindness, kidney failure, and limb amputations. Insulin-dependent diabetes mellitus (IDDM) accounts for 10 to 15% of all cases of diabetes mellitus.

Insulin injections are prescribed to the patients suffering from diabetes. Insulin is a natural hormone, which controls the level of the sugar glucose in the blood. In healthy people, insulin is released in blood by the pancreas as the concentration of blood glucose rises. Increased blood glucose levels, occur after meals and are rapidly compensated by a corresponding increase in insulin secretion. Insulin plays major role in converting the excess blood glucose into glycogen and storing it in liver.

Since the introduction of insulin in the 1920's, continuous strides have been made to improve the treatment of diabetes mellitus. To help avoid extreme glycaemia levels, diabetic patients often practice multiple injection therapy, whereby insulin is administered with each meal.

Insulin is a polypeptide of 51 amino acids, which are divided into 2 amino acid chains: the A chain having 21 amino acids and the B chain having 30 amino acids. The chains are connected to one another by means of two disulfide bridges. Insulin preparations have been employed for diabetes therapy for many years.

Traditionally short acting regular Insulin formulations or its intermediate acting Insulin Protamine formulations were used for treating patients with diabetes mellitus. With time, new insulin analogues and derivatives were developed. Insulin analogues and derivatives differ from human insulin at one or more than one amino acid positions and/or amino acid chain length.

A number of insulin, insulin analogs and derivatives are available in the market. The commonly used types of insulin, insulin analogs or insulin derivatives are categorized as: Rapid-acting Insulin analogs (Bolus): For example insulin aspart (Novolog®); Insulin lispro (Humalog®), Insulin Glulisine (Aprida®), Rapid acting human insulin (Viaject®). These analogs begin to work within 5 to 15 minutes of administration and are active for 3 to 4 hours.

Short-acting insulin (Bolus): For example Regular insulin (Humulin® or Novolin®). Regular insulin starts working within 30 minutes after administration and duration of action lasts from about 5 to 8 hours.

Intermediate-acting insulin: For example as Isophane insulin, Aspart protamine, Lispro protamine. It starts working in 1 to 3 hours after administration. Its duration of action varies between 16 to 24 hours.

Long-acting Insulin (Basal): For example Insulin glargine and Insulin detemir. Both these analogs starts working within 1 to 2 hours and their duration of action varies from about 12 to about 24 hours.

Mixed Insulin's: For example mixture of NPH and regular insulin. There are several variations with different proportions of the mixed insulin's. The onset of action of these mixed preparations is about 30 minutes. The mixed insulin's comprise same type of insulin. Two different types of insulin's cannot be mixed i.e. insulin lispro cannot be mixed with insulin detemir, insulin aspart or insulin glargine. The mixed formulation of insulin Lispro can only comprise insulin lispro regular and insulin lispro protamine two forms of insulin lispro.

Insulin analogs having an accelerated onset of action are described in EP0214826, EP0375437 and EP0678522. EP0124826 relates, inter alia, to substitutions of B27 and B28. EP0678522 describes insulin analogs, which have various amino acids, preferably proline, in position B29, but not glutamic acid.

EP0375437 includes insulin analogs with lysine or arginine in B28, which can optionally additionally be modified in B3 and/or A21. In EP0419504, insulin analogs are disclosed which are protected against chemical modifications, in which asparagine in B3 and at least one further amino acid in the positions A5, A15, A18 or A21 are modified. In WO 92/00321, insulin analogs are described in which at least one amino acid of the positions B1-B6 is replaced by lysine or arginine. According to WO92/00321, insulin's of this type have a prolonged action.

Ideally, exogenous insulin is administered at times and in doses that would yield a plasma profile, which mimics the plasma profile of endogenous insulin in a normal individual. The insulin preparations of naturally occurring insulin on the market for insulin substitution differ in the origin of the insulin (e.g. bovine, porcine, human insulin, or another mammalian or animal insulin), and also the composition, whereby the profile of action (onset of action and duration of action) can be influenced. Plasma profile of endogenous insulin can be attainted by combination of various insulin preparations. Preparations of naturally occurring insulin's, as well as preparations of insulin derivatives or insulin analogs which show modified kinetics, have been on the market for some time. Generally basal insulin's are given along with the bolus insulin's in order to mimic the normal endogenous plasma profile of insulin and to offer better control of post-prandial sugar levels.

There are two basal insulin's available in market Insulin glargine and Insulin detemir. Insulin glargine Gly(A21)-Arg(B31)-Arg(B32)-human insulin, a basal insulin, has a prolonged duration of action. It is injected once daily and is distinguished compared with other long-acting insulin's by its flat serum profile and the reduction of the danger of nightly hypoglycemia associated therewith (Schubert-Zsilavecz et al., 2: 125-130(2001)).

It is marketed under the trade name of Lantus by Sanofi Aventis. Lantus is injected as an acidic, clear solution and precipitates on account of its solution properties in the physiological pH range of the subcutaneous tissue as a stable hexamer associate. Two Formulations of Lantus are available in market
3 ml Cartridge Composition
10 ml Vial Composition

The 3 ml Cartridge composition comprises 100 IU (3.6378 mg) Insulin glargine, zinc, m-cresol, glycerol, and water for injection. The pH of the composition is adjusted to pH 4.0. The 10 ml vial composition comprises 100 IU Insulin glargine, zinc, m-cresol, glycerol, and water for injection. The pH of the composition is adjusted to pH 4.0 by addition of aqueous solutions of hydrochloric acid and sodium hydroxide.

The fast acting insulin's include Insulin Lispro, Insulin aspart and Insulin Glulisine. Fast acting insulin's that begins to work very quickly i.e. with 30 min of administration. Thus, they are used to control post-prandial increase in the sugar levels efficiently. These analogs are used to treat type 1 (insulin-dependent) diabetes and type 2 (non-insulin-dependent) diabetes. Insulin aspart is usually given together with another long-acting insulin.

The marketed composition of NovoLog® comprises 100 IU insulin aspart, glycerin, phenol, metacresol, zinc, disodium hydrogen phosphate dihydrate, sodium chloride and water for injection. The pH of the composition is adjusted to pH 7.2 to 7.6.

The marketed composition of Humalog® comprises 100 IU Insulin Lispro, glycerin, dibasic sodium phosphate, metacresol, zinc oxide, phenol, and Water for injection. The pH of the composition is adjusted between pH 7.0-8.0.

The marketed composition of Aprida® comprises 100 IU insulin glulisine, metacresol, tromethamine, sodium chloride, polysorbate 20, and Water for injection. The pH of the composition is adjusted to pH 7.3.

Novo Nordisk has formulated a soluble co-formulation of long acting basal insulin, insulin degludec and the rapid acting insulin analogue, insulin aspart (B28Asp human insulin). Ryzodeg is the first fully soluble ready to use insulin product for subcutaneous (s.c.) injection.

Basal-bolus insulin therapy given as either multiple daily injections or by an insulin pump is a mainstay of diabetes treatment for achieving optimal glycemic control in type 1 diabetes. Attempts have been made to mix the different types of insulin in one injection in order to reduce the number of injections to be administered to a patient in the day. Several warnings have been issued by the innovators of rapid acting insulin's and basal insulin against mixing long- and rapid-acting insulin's together (insulin glargine, Lantus; sanofi-aventis; sanofi-aventis, available from http://www.lantus.com/hcp/closing.aspx; insulin detemir rDNA origin, brand name Levemir drug insert; Novo Nordisk, Bagsvaerd, Denmark).

Despite warnings from manufacturers of insulin formulations, some patients and pediatric practitioners mixed the basal insulin's and bolus insulin's in one syringe just before injection. The reason for such warnings is that when insulin's of different isoelectric point (pI) insulin's are mixed in a syringe, it results in uncontrolled precipitation and it is difficult to predict the exact dose and the time of action of the precipitated mixture.

The known rapid acting insulin analogs and unmodified insulin have pI between 5-5.5 and thus the formulations comprising them in solution form is in basic range i.e. 7.0 to 8.0, whereas the pI of Insulin glargine is above 6.5 and thus the formulation of Lantus is in acidic range i.e. pH 3.8-4.2. Glargine cannot be mixed with any other kind of insulin such as short acting or rapid acting insulin's because the pI of the glargine is different from other insulin analogs or derivatives. None of the prior arts discloses a stable formulation comprising combination of the insulin's having different isoelectric points comprising readily dissociable molecular aggregates of defined particle size.

There are number of prior arts disclosing the combination/mixing of basal insulin's with rapid acting insulin analogs and pharmaceutical compositions comprising the combination.

Kaplan W et al. (2004) Effects of mixing glargine and short-acting insulin analogs on glucose control. The author concluded that mixing glargine with lispro or aspart insulin in the same syringe and dividing the dose of glargine or twice-daily dosing does not affect short-term glycemic profile. (Diabetes Care. 2004 November; 27(11):2739-40)

Fiallo-Scharer R et al. (2006) disclosed that there were no significant differences in glycemic control between children who mixed Insulin glargine in the same syringe with a Rapid acting insulin analogue compared with children who took separate injections. (J Pediatr. 2006 April; 148(4):481-4.)

Hassan K et al. (2008) discloses that glycemic control with insulin glargine mixed with a rapid-acting insulin analog given twice daily seems significantly more effective than the standard therapy in newly diagnosed type 1 diabetes. Furthermore, it decreases pain and burden of injections for children with diabetes by allowing patients to mix glargine with rapid-acting insulin analog. (Pediatrics 2008; 121: e466 e472.)

Evans et al. (2011) discloses that neither insulin glargine nor insulin detemir are suitable for mixing with other insulin analogues as this mixing substantially alters their pharmacokinetic properties.

Nicolucci, (2011) Antonio et al. discloses optimization of basal insulin glargine with one bolus injection of insulin glulisine given separately. (Diabetes Care (2011), 34(12), 2524-2526)

Al Shamsi A. M et. al. (2008) discloses that glycemic control with insulin glargine (IG) mixed with a rapid-acting insulin analog given twice daily seems significantly more effective than the standard therapy in newly diagnosed type 1 diabetes. (Pediatrics. 2008 September; 122 (3):675-6; author reply 676)

Cengiz E et. al (2010) discloses mixing insulin lispro with insulin glargine markedly flattens the early pharmacodynamic peak of lispro and causes a shift to the right in the Glucose Infusion Rate (GIR) curve changes that might lead to difficulties in controlling meal-related glucose excursions. (Diabetes Care 2010; 33:1009-1012.)

Lucchesi Mb et. al. (2012) discloses mixing Insulin Lispro with Insulin glargine immediately before the Suncutaneous injection decreases Insulin Lispro serum peak concentration without affecting the glycemic profile after 12 wk in this group with type 1 diabetes mellitus. (Dib SA.2012 November; 13(7):519-24. doi: 10.1111/j.1399-5448.2012.00867)

Thanh M. N. et. al. (2010) discloses insulin detemir mixed with insulin aspart given twice daily had equivalent effects on blood glucose when compared with giving insulin detemir and insulin aspart as separate injections twice daily in children with type 1 diabetes. There was no increase in hypoglycemia in either treatment. (Diabetes Care. 2010 August; 33(8): 1750-1752.)

Cengiz E et. al. (2012) discloses that mixing insulin aspart with insulin detemir markedly lowers the early pharmacodynamic action of insulin aspart and prolongs its time-action profile as compared with the separate injection of these analogs. (Diabetes Care. 2012 April; 35(4):690-2)

U.S. Pat. Nos. 7,713,929 and 7,718,609 discloses a composition comprising a rapid or intermediate acting insulin in combination with a long acting insulin, wherein the pH of the composition is adjusted to a pH of between 3.8 and 4.2 to solubilize the long acting insulin.

U.S. Pat. No. 8,084,420 discloses an injectable formulation comprising a fast acting, rapid acting or very rapid acting insulin including a chelator and a dissolution agent in combination with an intermediate acting in a form suitable for subcutaneous administration. The patent discloses that Lantus™ (insulin glargine) was mixed with Viaject™ (rapid acting insulin's) and the compatibility of these formulations is due to similarity in pH of Lantus and Viaject compositions. The '420 patent discloses such compositions when administered to patient, the Lantus™ had shorter duration of action, when given in single injection along with Viaject™.

US Patent Application No. 20130065826 discloses a composition in the form of an injectable aqueous solution, the pH of which is between 6.0 and 8.0, comprising at least: a) a basal insulin, the isoelectric point pI of which is between 5.8 and 8.5; and b) a modified dextran polymer. The composition may additionally comprise prandial insulin.

PCT Publication Nos. 2009021956A1 and 2009021955A1 discloses a pharmaceutical composition comprising the new fast acting insulin analogues in mixture with long action insulin analogues. The mixibility with long acting insulin analogues is achieved through substitution of Zn-binding His in position B-10 of human insulin with Ile, Val, Ala or Phe.

PCT Publication No. 2007041481 discloses an injection of insulin glargine at a dose equivalent to the subject's usual daily dose of basal insulin mixed with VIAJECT™.

PCT Publication No. 2011094632 discloses in various embodiments the one or more insulin analogs include a combination of both a long-acting insulin analog in combination with a fast-acting insulin analog.

None of the prior arts discloses a premixed ready-to-use biphasic pharmaceutical composition comprising readily dissociable molecular aggregates formed by combining an insulin analogue or derivative having isoelectric point between 5.8 to 8.5 in combination with one or more insulin analogue or derivative having isoelectric point between 4.0 to 5.7 optionally along with one or more excipients, wherein the said particle aggregates have an average particle size between about 5 μm to about 20 μm. The formulation does not involve any without any chemical modification in insulin chains to make the two insulin's compativle. Insulin glargine has isoelectric point (pI) at pH 6.8.

Thus, insulin glargine is soluble at acidic pH and precipitates at neutral pH. On the other hand all other rapid, short or intermediate acting insulin's have isoelectric point at pH 5.5-5.8 and remain solubilized at neutral pH. Thus, formulating a pharmaceutical composition, wherein the active agents has different pI is a challenge for a formulator. Further, insulin's are known to be highly sensitive molecule, wherein little change in the formulation causes a profound change in the surface morphology and molecular conformation, which can alter its activity. Thus, there is a need for pharmaceutical composition wherein insulin's with different isoelectric points can be mixed in order to have stable, bioavailable formulations wherein the release can be controlled and defined.

### SUMMARY OF THE INVENTION

As used herein the term “readily dissociable molecular aggregates” means loosely bound molecular aggregates which dissociate immediately into individual particles on shaking or on in-vivo administration of the formulation

As used herein the term “rapid or short acting insulin” means type of insulin's which are rapidly absorbed in <30 minutes following injection and have a short time to peak insulin concentration of 1 hour and a shorter duration of action of 3-4 hours when compared with regular human insulin.

As used herein the term “intermediate acting insulin” means type of insulin's that starts to lower blood glucose within 1 to 2 hours after injection and has its strongest effect from 6 to 12 hours.

As used herein the term “pharmaceutical composition” means a mixture containing a therapeutic compound to be administered to a mammal, e.g., a human, in order to prevent, treat or control a particular disease or condition affecting the mammal.

The term “buffer” used herein means a solution containing either a weak acid and its salt or a weak base and its salt, which is resistant to changes in pH.

By “preservative” as used herein refers to the compound that can be used to prevent the growth of fungi and other microorganisms.

By “isotonic agent” as used herein refers to a compound that is physiologically tolerated and imparts a suitable tonicity to a formulation to prevent the net flow of water across cell membranes that are in contact with the formulation.

By “pH modifying agent” as used herein refers to a combination of acid and alkali.

By “solubilizing agent” as used herein refers to a material able to solubilize or partially solubilize the therapeutic compound and/or polymer.

By “chelator” as used herein refers to a ligand that can form a chelate with a metal atom.

By “acidifying agent” as used herein refers to a chemical species that donates protons or hydrogen ions and/or accepts electrons.

By “devoid of chelator” as used herein is meant said chelator is present in a concentration less than 0.01% w/v of final formulation.

By “devoid an acidifying agent” as used herein is meant said acidifying agent is present in a concentration less than 0.01% w/v of final formulation.

By “devoid of dextran” as used herein is meant said dextran polymer is not present in the formulation.

One of the aspects of the present invention provides a pharmaceutical composition comprising readily dissociable molecular aggregates formed by combining an insulin, insulin analogue, derivative or metabolite having isoelectric point between 5.8 to 8.5 with one or more an insulin, insulin analogue, derivative or metabolite having isoelectric point between 4.0 to 5.7 optionally along with one or more excipients, wherein the said molecular aggregates have an average particle size between about 5 μm to about 20 μm.

Another aspect of the present invention provides a method for treating Type I and Type II Diabetes Mellitus in a patient comprising administering to said patient a pharmaceutical comprising readily dissociable molecular aggregates formed by combining an insulin, insulin analogue, derivative or metabolite having isoelectric point between 5.8 to 8.5 with one or more an insulin, insulin analogue, derivative or metabolite having isoelectric point between 4.0 to 5.7 optionally along with one or more excipients, wherein the said molecular aggregates have an average particle size between about 5 μm to about 20 μm.

Another aspect of the present invention provides a method of extending the duration of exposure of a long acting insulin in the treatment of Type I and Type II Diabetes Mellitus in a patient comprising administering to said patient comprising readily dissociable molecular aggregates formed by combining an insulin, insulin analogue, derivative or metabolite having isoelectric point between 5.8 to 8.5 with one or more an insulin, insulin analogue, derivative or metabolite having isoelectric point between 4.0 to 5.7 optionally along with one or more excipients, wherein the said molecular aggregates have an average particle size between about 5 μm to about 20 μm.

Another aspect of the present invention provides a method of reducing the nocturnal hypoglycemia, wherein nocturnal hypoglycemia in patients suffering from Type I or Type II diabetes, wherein the method comprise administering to the said patient comprising readily dissociable molecular aggregates formed by combining an insulin, insulin analogue, derivative or metabolite having isoelectric point between 5.8 to 8.5 with one or more an insulin, insulin analogue, derivative or metabolite having isoelectric point between 4.0 to 5.7 optionally along with one or more excipients, wherein the said molecular aggregates have an average particle size between about 5 μm to about 20 μm.

Another aspect of the present invention provides a method for obtaining a flattened insulin plasma concentration to time profile in a patient, wherein the method comprises administering to a patient in need thereof, a pharmaceutical composition comprising readily dissociable molecular aggregates formed by combining an insulin, insulin analogue, derivative or metabolite having isoelectric point between 5.8 to 8.5 in combination with one or more an insulin, insulin analogue, derivative or metabolite having isoelectric point between 4.0 to 5.7 optionally along with one or more excipients, wherein the said molecular aggregates have an average particle size between about 5 μm to about 20 μm.

Another aspect of the present invention provides a process for preparing a pharmaceutical composition comprising readily dissociable molecular aggregates formed by combining an insulin, insulin analogue, derivative or metabolite having isoelectric point between 5.8 to 8.5 with one or more an insulin, insulin analogue, derivative or metabolite having isoelectric point between 4.0 to 5.7 optionally along with one or more excipients, wherein the said molecular aggregates have an average particle size between about 5 μm to about 20 μm, wherein the process comprises
a) preparing a pharmaceutical composition comprising 100 IU-1000 IU of an insulin, insulin analogue, derivative or metabolite having isoelectric point between 5.8 to 8.5, wherein the pH of the composition is 3.0-4.5.
b) preparing a pharmaceutical composition comprising 100 IU-1000 IU of one or more rapid, short or intermediate acting insulin, wherein the pH of the composition is 6.0-8.0.
c) mixing the an insulin, insulin analogue, derivative or metabolite having isoelectric point between 5.8 to 8.5 composition of step a) with the composition of rapid, short or intermediate acting insulin of step b) such that the final pH of the composition is pH 6.5-8.0.

One of the aspects of the present invention provides a pharmaceutical composition comprising readily dissociable molecular aggregates formed by combining an insulin, insulin analogue, derivative or metabolite having isoelectric point between 5.8 to 8.5 with one or more an insulin, insulin analogue, derivative or metabolite having isoelectric point between 4.0 to 5.7, one or more stabilizing agents optionally along with one or more excipients or combination thereof, wherein the said molecular aggregates have an average particle size between about 5 μm to about 20 μm.

### DETAILED DESCRIPTION OF THE INVENTION

While developing a formulation comprising combination of insulin glargine with other insulin analogs, inventors of the present application have surprisingly found that when insulin glargine is formulated with the one or more rapid, short or intermediate acting insulin's, wherein the pH of the composition is adjusted to a pH of between 6.0 and 8.0; the resultant formulation is stable. The present formulations are easy to manufacture and are ready-to-use. Further, the content uniformity of the formulation can be achieved by simultaneously mixing the two different formulations at the time of filling. The present formulations produce anti-diabetic effect for prolonged period of time i.e. more than 24 h. The present invention has extended duration of effect.

One of the aspects of the present invention provides a pharmaceutical composition comprising readily dissociable molecular aggregates formed by combining an insulin, insulin analogue, derivative or metabolite having isoelectric point between 5.8 to 8.5 with one or more an insulin, insulin analogue, derivative or metabolite having isoelectric point between 4.0 to 5.7 optionally along with one or more excipients or combination thereof, wherein the said molecular aggregates have an average particle size between about 5 μm to about 20 μm and the pH of the composition is adjusted to a pH of between 3.0 and 8.0.

One of the aspects of the present invention provides a pharmaceutical composition comprising readily dissociable molecular aggregates formed by combining an insulin, insulin analogue, derivative or metabolite having isoelectric point between 5.8 to 8.5 with one or more an insulin, insulin analogue, derivative or metabolite having isoelectric point between 4.0 to 5.7 optionally along with one or more excipients, wherein the said molecular aggregates have an average particle size between about 5 μm to about 20 μm and the pH of the composition is adjusted to a pH of between 6.0 and 8.0.

One of the aspects of the present invention provides a pharmaceutical composition comprising readily dissociable molecular aggregates formed by combining 100 IU to 1000 IU of an insulin, insulin analogue, derivative or metabolite having isoelectric point between 5.8 to 8.5 with 100 IU to 1000 IU of one or more an insulin, insulin analogue, derivative or metabolite having isoelectric point between 4.0 to 5.7 optionally along with one or more excipients, wherein the said molecular aggregates have an average particle size between about 5 μm to about 20 μm.

One or more insulin, insulin analogues, derivatives or metabolites are present in concentration range of 40 IU to 1000 IU per ml.

One or more insulin analogue or derivative having isoelectric point between 4.0 to 5.7 according to present invention comprises recombinant human insulin, Insulin NPH, Insulin Lispro, Insulin Lispro Protamine, Insulin Glulisine and Insulin Aspart, Insulin Aspart Protamine, A21 Gly Insulin, A21Gly B28Lys insulin, A21Gly B28Lys B29Pro, A21Gly B28Asp or Viaject (rapid acting insulin).

The insulin analogue or derivative having isoelectric point between 4.0 to 5.7 is Insulin aspart.

The insulin analogue or derivative having isoelectric point between 4.0 to 5.7 is A21 Gly Insulin.

One or more insulin, insulin analogue, derivative or metabolite having isoelectric point between 5.8 to 8.5 comprises insulin glargine or A21Gly B31Arg insulin.

One or more insulin, insulin analogue, derivative or metabolite having isoelectric point between 5.8 to 8.5 is insulin glargine.

The compositions of present invention are biphasic composition.

One of the aspects of the present invention provides a pharmaceutical composition comprising readily dissociable molecular aggregates formed by combining 100 IU to 1000 IU of an insulin, insulin analogue, derivative or metabolite having isoelectric point between 5.8 to 8.5 with 100 IU to 1000 IU of one or more an insulin, insulin analogue, derivative or metabolite having isoelectric point between 4.0 to 5.7 optionally along with one or more excipients, wherein the said molecular aggregates have an average particle size between about 5 μm to about 20 μm, wherein the pH of the composition is between 6.0 to 8.0.

One of the aspects of the present invention provides a pharmaceutical composition comprising readily dissociable molecular aggregates formed by combining 100 IU to 1000 IU of an insulin, insulin analogue, derivative or metabolite having isoelectric point between 5.8 to 8.5 with 100 IU to 1000 IU of one or more an insulin, insulin analogue, derivative or metabolite having isoelectric point between 4.0 to 5.7 optionally along with one or more excipients, wherein the said molecular aggregates have an average particle size between about 5 μm to about 20 μm, wherein the pH of the composition is between 6.5 to 7.5.

One of the aspects of the present invention provides a pharmaceutical composition comprising readily dissociable molecular aggregates formed by combining insulin glargine with one or more an insulin, insulin analogue, derivative or metabolite having isoelectric point between 4.0 to 5.7 selected from the group consisting insulin aspart, Insulin Lispro, Insulin Glulisine or A21 Gly insulin optionally along with one or more excipients, wherein the said molecular aggregates have an average particle size between about 5 μm to about 20 μm.

Another aspect of the present invention provides a pharmaceutical composition comprising readily dissociable molecular aggregates formed by combining insulin glargine with one or more an insulin, insulin analogue, derivative or metabolite having isoelectric point between 4.0 to 5.7 selected from the group consisting insulin aspart, Insulin Lispro, Insulin Glulisine or A21 Gly insulin optionally along with one or more excipients, wherein the said molecular aggregates have an average particle size between about 5 μm to about 20 μm.

Another aspect of the present invention provides a pharmaceutical composition comprising readily dissociable molecular aggregates formed by combining insulin glargine with insulin aspart, wherein the said molecular aggregates have an average particle size between about 5 μm to about 20 μm.

Another aspect of the present invention provides a pharmaceutical composition comprising readily dissociable molecular aggregates formed by combining insulin glargine with A21Gly Insulin, wherein the said molecular aggregates have an average particle size between about 5 μm to about 20 μm.

Another aspect of the present invention involves a pharmaceutical composition comprising readily dissociable molecular aggregates formed by combining 100 IU 1000 IU of insulin glargine with 100 IU 1000 IU of Insulin Aspart, wherein the said molecular aggregates have an average particle size between about 5 μm to about 20 μm.

Another aspects of the present invention involves a pharmaceutical composition comprising readily dissociable molecular aggregates formed by combining 100 IU 1000 IU of insulin glargine with 100 IU 1000 IU of Insulin Lispro, wherein the said molecular aggregates have an average particle size between about 5 μm to about 20 μm

Another aspects of the present invention involves a pharmaceutical composition comprising readily dissociable molecular aggregates formed by combining 100 IU 1000 IU of insulin glargine with 100 IU 1000 IU of Insulin Glulisine, wherein the said molecular aggregates have an average particle size between about 5 μm to about 20 μm

Another aspects of the present invention involves a pharmaceutical composition comprising readily dissociable molecular aggregates formed by combining 100 IU 1000 IU of insulin glargine with 100 IU 1000 IU of A21Gly insulin, wherein the said molecular aggregates have an average particle size between about 5 μm to about 20 μm

One of the aspects of the present invention provides a pharmaceutical composition comprising readily dissociable molecular aggregates formed by combining 100 IU to 1000 IU of an insulin, insulin analogue, derivative or metabolite having isoelectric point between 5.8 to 8.5 with 100 IU to 1000 IU of one or more an insulin, insulin analogue, derivative or metabolite having isoelectric point between 4.0 to 5.7 optionally along with one or more excipients, wherein the said molecular aggregates have an average particle size between about 5 μm to about 20 μm, wherein the pH of the composition is adjusted to a pH of between 6.0 and 8.0.

To reduce the volume of injection, a formulation containing 100 IU-1000 IU of an insulin, insulin analogue, derivative or metabolite having isoelectric point between 5.8 to 8.5 and 100 IU-1000 IU of an insulin, insulin analogue, derivative or metabolite having isoelectric point between 4.0 to 5.7 in 1 ml has been developed. Thus, the total amount of insulin's i.e. insulin, insulin analogue, derivative or metabolite having isoelectric point between 5.8 to 8.5 and an insulin, insulin analogue, derivative or metabolite having isoelectric point between 4.0 to 5.7 present in the formulation is between 200 to 1000 IU/ml. As the formulation involves combination of insulin, insulin analogue, derivative or metabolite having isoelectric point between 5.8 to 8.5 and an insulin, insulin analogue, derivative or metabolite having isoelectric point between 4.0 to 5.7 in one vial with reduced volume for injection, the formulation requires fewer injection per day. This leads to increased patient compliance.

One of the aspects of the present invention provides a pharmaceutical composition comprising readily dissociable molecular aggregates formed by combining 100 IU to 1000 IU of an insulin, insulin analogue, derivative or metabolite having isoelectric point between 5.8 to 8.5 with 100 IU to 1000 IU of one or more an insulin, insulin analogue, derivative or metabolite having isoelectric point between 4.0 to 5.7 optionally along with one or more excipients, wherein the said molecular aggregates have an average particle size between about 5 μm to about 20 μm, wherein the pH of the composition is adjusted to a pH of between 6.8 and 7.2.

The insulin, insulin analogue, derivative or metabolite having isoelectric point between 5.8 to 8.5 and one or more an insulin, insulin analogue, derivative or metabolite having isoelectric point between 4.0 to 5.7 is present in the pharmaceutical composition in ratio 1:99 to 99:1.

One of the aspects of the present invention provides a pharmaceutical composition comprising readily dissociable molecular aggregates formed by combining 100 IU to 1000 IU of an insulin, insulin analogue, derivative or metabolite having isoelectric point between 5.8 to 8.5 with 100 IU to 1000 IU of one or more an insulin, insulin analogue, derivative or metabolite having isoelectric point between 4.0 to 5.7 optionally along with one or more excipients, wherein the said molecular aggregates have an average particle size between about 5 μm to about 20 μm, wherein the wherein the composition is devoid of chelator.

Suitable chelators, as used herein include, ethylenediaminetetraacetic acid (EDTA), citric acid, dimercaprol (BAL), penicillamine, alginic acid, chlorella, cilantro, alpha lipoic acid, dimercaptosuccinic acid (DMSA), dimercaptopropane sulfonate (DMPS), and oxalic acid.

One of the aspects of the present invention provides a pharmaceutical composition comprising readily dissociable molecular aggregates formed by combining 100 IU to 1000 IU of an insulin, insulin analogue, derivative or metabolite having isoelectric point between 5.8 to 8.5 with 100 IU to 1000 IU of one or more an insulin, insulin analogue, derivative or metabolite having isoelectric point between 4.0 to 5.7 optionally along with one or more excipients, wherein the said molecular aggregates have an average particle size between about 5 μm to about 20 μm, wherein the wherein the composition is devoid of acidifying agent.

Suitable acidifying agents include formic acid, ascorbic acid, aspartic acid, benzene sulphonic acid, benzoic acid, hydrochloric acid, sulphuric acid, phosphoric acid, nitric acid, tartaric acid, diatrizoic acid, glutamic acid, lactic acid, maleic acid, succinic acid, acetic acid, citric acid or anhydrous citric acid, including such agents in particulate solid form.

One of the aspects of the present invention provides a pharmaceutical composition comprising readily dissociable molecular aggregates formed by combining 100 IU to 1000 IU of an insulin, insulin analogue, derivative or metabolite having isoelectric point between 5.8 to 8.5 with 100 IU to 1000 IU of one or more an insulin, insulin analogue, derivative or metabolite having isoelectric point between 4.0 to 5.7 optionally along with one or more excipients, wherein the said molecular aggregates have an average particle size between about 5 μm to about 20 μm, wherein the wherein the composition is devoid of dextran polymer.

Dextran polymer means dextran polymers claimed in US Patent Application 20130065826 are included herein.

One of the aspects of the present invention provides a method for obtaining a flattened insulin plasma concentration to time profile in a patient, wherein the method comprises administering to a patient in need thereof, a pharmaceutical composition comprising readily dissociable molecular aggregates formed by combining an insulin, insulin analogue, derivative or metabolite having isoelectric point between 5.8 to 8.5 in combination with one or more an insulin, insulin analogue, derivative or metabolite having isoelectric point between 4.0 to 5.7 optionally along with one or more excipients, wherein the said molecular aggregates have an average particle size between about 5 μm to about 20 μm, wherein the composition is administered through subcutaneous, intramuscular or intravenous route.

Another aspect of the present invention provides a method for obtaining a flattened insulin plasma concentration to time profile in a patient, wherein the method comprises administering to a patient in need thereof, a pharmaceutical composition comprising readily dissociable molecular aggregates formed by combining an insulin, insulin analogue, derivative or metabolite having isoelectric point between 5.8 to 8.5 in combination with one or more an insulin, insulin analogue, derivative or metabolite having isoelectric point between 4.0 to 5.7 optionally along with one or more excipients, wherein the said molecular aggregates have an average particle size between about 5 μm to about 20 μm, wherein the composition is administered subcutaneously.

The pharmaceutical compositions of the present invention are liquid or lyophilized formulations.

The pharmaceutical compositions of the present invention are for oral, transmucosal, nasal or parenteral administration.

The pharmaceutical compositions of the present invention are for parenteral administration.

The pharmaceutical composition of present invention is administered as subcutaneous or intravenous injection or intravenous infusion.

In one aspect of the present invention, there is provided a pharmaceutical composition comprising readily dissociable molecular aggregates formed by combining an insulin, insulin analogue, derivative or metabolite having isoelectric point between 5.8 to 8.5 with one or more an insulin, insulin analogue, derivative or metabolite having isoelectric point between 4.0 to 5.7 along with one or more excipients, wherein the said molecular aggregates have an average particle size between about 5 μm to about 20 μm, wherein the excipients selected from the group consisting of isotonic agent, surfactant, buffer, zinc or salt thereof, preservatives, pH modifying agents, stabilizing agents, solubilizing agents and the combination of the aforementioned excipients.

In one aspect of the present invention, there is provided a pharmaceutical composition comprising readily dissociable molecular aggregates formed by combining 100 IU 1000 IU of an insulin, insulin analogue, derivative or metabolite having isoelectric point between 5.8 to 8.5 with 100 IU 1000 IU of one or more an insulin, insulin analogue, derivative or metabolite having isoelectric point between 4.0 to 5.7 along with one or more excipients, wherein the said molecular aggregates have an average particle size between about 5 μm to about 20 μm, wherein the excipients selected from the group consisting of isotonic agent, surfactant, buffer, zinc or salt thereof, preservatives, pH modifying agents, stabilizing agents and solubilizing agents.

The excipients include, but are not limited to, isotonic agent, surfactant, buffer, zinc or salt thereof, preservatives, pH modifying agents, solubilizing agents, stabilizing agents and combination thereof.

An “isotonic agent” is a compound, such as glycerin, are commonly used for such purposes at known concentrations. Other possible isotonicity agents include salts, e.g., sodium chloride, dextrose, or lactose.

An “surfactant” as used herein include, but are not limited to, partial and fatty acid esters and ethers of polyhydric alcohols such as of glycerol, sorbitol and the like (Span®, Tween®, in particular Tween® 20 and Tween®80, Myrj®, Brij®, Cremophore® or poloxamers, Pluronics® and Tetronics®), polysorbates (Tween™), sodium dodecyl sulfate (sodium lauryl sulfate), lauryl dimethyl amine oxide, cetyltrimethylammonium bromide (CTAB), polyethoxylated alcohols polyoxyethylene sorbitan, Octoxynol (Triton X100™), N, N-dimethyldodecylamine-N-oxide, hexadecyltrimethylammonium bromide (HTAB), polyoxyl 10 lauryl ether, Brij 721™, bile salts (sodium deoxycholate, sodium cholate), polyoxyl castor oil (Cremophor™), nonylphenol ethoxylate (Tergitol™), cyclodextrins, lecithin, and methylbenzethonium chloride (Hyamine™).

An “buffer” as used herein include, but are not limited to, phosphate, acetate, citrate, arginine, glycylglycine or TRIS (i.e. 2-amino-2-hydroxymethyl-1,3-propanediol) buffer and corresponding salts.

The “preservatives” as used herein include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, 2-Penoxyethanol, Phenyl mercuric nitrate, Thimerosal, metacresol or combinations thereof.

An “pH modifying agents” as used herein can be selected form the group comprising of o-phosphoric acid, citric acid, acetic acid, succinic acid, lactic acid, gluconic acid, tartaric acid, 1,2,3,4-butane tetracarboxylic acid, fumaric acid or malic acid. Alkali is selected form the group comprising of sodium hydroxide, potassium hydroxide, sodium hydroxide, ammonium hydroxide, magnesium oxide, calcium hydroxide, calcium carbonate, magnesium carbonate, magnesium aluminum silicates, diethanolamine, monoethanolamine, sodium carbonate, sodium bicarbonate or triethanolamine.

An “antioxidants” as used herein can be selected from the group comprising of ascorbate (sodium/acid), bisulite sodium, butylated hydroxy anisole (bha), butylated hydroxy toluene (bht), cystein/cysteinate hcl, dithionite sodium (na hydrosulite, na sulfoxylate), gentisic acid, gentisic acid ethanolamine, glutamate monosodium, glutathione, formaldehyde sulfoxylate sodium, metabisulite potassium, metabisulite sodium, methionine, monothioglycerol (thioglycerol), propyl gallate, sulfite sodium, tocopherol alpha, alpha tocopherol hydrogen succinate, thioglycolate sodium or combination thereof.

An “solubilizing agents” as used herein include, but are not limited to, include wetting agents such as polysorbates and poloxamers, non-ionic and ionic surfactants, food acids and bases (e.g. sodium bicarbonate), polyhydric alcohols and alcohols.

The “stabilizing agents” as used herein include, but are not limited to surfactants, antioxidants, preservatives, solubilizing agents, esterase inhibitors and combination thereof. The stabilizing agents are selected from the group consisting of partial and fatty acid esters and ethers of polyhydric alcohols such as of glycerol, sorbitol and the like (Span®, Tween®, in particular Tween® 20 and Tween®80, Myrj®, Brij®, Cremophore® or poloxamers, Pluronics® and Tetronics®), polysorbates (Tween™), sodium dodecyl sulfate (sodium lauryl sulfate), lauryl dimethyl amine oxide, cetyltrimethylammonium bromide (CTAB), polyethoxylated alcohols polyoxyethylene sorbitan, Octoxynol (Triton X100™), N, N-dimethyldodecylamine-N-oxide, hexadecyltrimethylammonium bromide (HTAB), polyoxyl 10 lauryl ether, Brij 721™, bile salts (sodium deoxycholate, sodium cholate), polyoxyl castor oil (Cremophor™), nonylphenol ethoxylate (Tergitol™), cyclodextrins, lecithin, methylbenzethonium chloride (Hyamine™), benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, 2-Penoxyethanol, Phenyl mercuric nitrate, Thimerosal, metacresol, ascorbate (sodium/acid), bisulite sodium, butylated hydroxy anisole (BHA), butylated hydroxy toluene (BHT), cystein/cysteinate HCl, dithionite sodium (Na hydrosulite, Na sulfoxylate), gentisic acid, gentisic acid ethanolamine, glutamate monosodium, glutathione, formaldehyde sulfoxylate sodium, metabisulite potassium, metabisulite sodium, methionine, monothioglycerol (thioglycerol), propyl gallate, sulfite sodium, tocopherol alpha, alpha tocopherol hydrogen succinate, thioglycolate sodium, esterase inhibitors such as pancreatic secretary inhibitors, protease inhibitors, and serine esterase inhibitors such as aprotinin.

Another aspect of the present invention provides a method for treating Type I and Type II Diabetes Mellitus in a patient comprising administering to said patient a pharmaceutical comprising readily dissociable molecular aggregates formed by combining an insulin, insulin analogue, derivative or metabolite having isoelectric point between 5.8 to 8.5 with one or more an insulin, insulin analogue, derivative or metabolite having isoelectric point between 4.0 to 5.7 optionally along with one or more excipients, wherein the said molecular aggregates have an average particle size between about 5 μm to about 20 μm, wherein the composition is premixed ready to use biphasic formulation.

Another aspect of the present invention provides a method for treating Type I and Type II Diabetes Mellitus in a patient comprising administering to said patient a pharmaceutical comprising readily dissociable molecular aggregates formed by combining an insulin, insulin analogue, derivative or metabolite having isoelectric point between 5.8 to 8.5 with one or more an insulin, insulin analogue, derivative or metabolite having isoelectric point between 4.0 to 5.7 optionally along with one or more excipients, wherein the said molecular aggregates have an average particle size between about 5 μm to about 20 μm, wherein the pH of the composition is adjusted to a pH of between 6.5 and 8.0.

Another aspect of the present invention provides a method for treating Type I and Type II Diabetes Mellitus in a patient comprising administering to said patient a pharmaceutical comprising readily dissociable molecular aggregates formed by combining 100 IU-1000 IU of an insulin, insulin analogue, derivative or metabolite having isoelectric point between 5.8 to 8.5 with 100 IU-1000 IU of one or more an insulin, insulin analogue, derivative or metabolite having isoelectric point between 4.0 to 5.7 optionally along with one or more excipients, wherein the said molecular aggregates have an average particle size between about 5 μm to about 20 μm, wherein the pH of the composition is adjusted to a pH of between 6.5 and 8.0.

Another aspect of the present invention provides a method of extending the duration of exposure of a long acting insulin in the treatment of Type I and Type II Diabetes Mellitus in a patient comprising administering to said patient comprising readily dissociable molecular aggregates formed by combining 100 IU-1000 IU of an insulin, insulin analogue, derivative or metabolite having isoelectric point between 5.8 to 8.5 with 100 IU-1000 IU of one or more an insulin, insulin analogue, derivative or metabolite having isoelectric point between 4.0 to 5.7 optionally along with one or more excipients, wherein the said molecular aggregates have an average particle size between about 5 μm to about 20 μm.

Another aspect of the present invention provides a method of extending the duration of exposure of a long acting insulin in the treatment of Type I and Type II Diabetes Mellitus in a patient comprising administering to said patient comprising readily dissociable molecular aggregates formed by combining 100 IU-1000 IU of an insulin, insulin analogue, derivative or metabolite having isoelectric point between 5.8 to 8.5 with 100 IU-1000 IU of one or more an insulin, insulin analogue, derivative or metabolite having isoelectric point between 4.0 to 5.7 optionally along with one or more excipients, wherein the said molecular aggregates have an average particle size between about 5 μm to about 20 μm and having pH between 6.0 to 8.0.

In one aspect of the present invention, there is provided a process for preparing a pharmaceutical composition comprising readily dissociable molecular aggregates formed by combining an insulin, insulin analogue, derivative or metabolite having isoelectric point between 5.8 to 8.5 with one or more an insulin, insulin analogue, derivative or metabolite having isoelectric point between 4.0 to 5.7 optionally along with one or more excipients, wherein the said molecular aggregates have an average particle size between about 5 μm to about 20 μm, wherein the process comprises
a) preparing a pharmaceutical composition comprising 100 IU-1000 IU of Insulin Glargine, wherein the pH of the composition is 3.0-4.5.
b) preparing a pharmaceutical composition comprising 100 IU-1000 IU of one or more rapid, short or intermediate acting insulin, wherein the pH of the composition is 6.0-8.0.
c) mixing the insulin glargine composition of step a) with the composition of rapid, short or intermediate acting insulin of step b) such that the final pH of the composition is pH 6.5-8.0.

In one aspect of the present invention, there is provided a process for preparing a a pharmaceutical composition comprising readily dissociable molecular aggregates formed by combining an insulin, insulin analogue, derivative or metabolite having isoelectric point between 5.8 to 8.5 with one or more an insulin, insulin analogue, derivative or metabolite having isoelectric point between 4.0 to 5.7 optionally along with one or more excipients, wherein the said molecular aggregates have an average particle size between about 5 μm to about 20 μm, wherein the process comprises
a) preparing a pharmaceutical composition comprising 100 IU-1000 IU of Insulin Glargine, wherein the pH of the composition is 3.0-4.5.
b) preparing a pharmaceutical composition comprising 100 IU-1000 IU of one or more rapid, short or intermediate acting insulin, wherein the pH of the composition is 6.0-8.0.
c) simultaneously mixing the insulin glargine composition of step a) with the composition of rapid, short or intermediate acting insulin of step b) at the time of filling into the vial or cartridge such that the final pH of the composition is pH 6.5-8.0.

In one aspect of the present invention, there is provided a process for preparing a pharmaceutical composition comprising readily dissociable molecular aggregates formed by combining an insulin, insulin analogue, derivative or metabolite having isoelectric point between 5.8 to 8.5 with one or more an insulin, insulin analogue, derivative or metabolite having isoelectric point between 4.0 to 5.7 optionally along with one or more excipients, wherein the said molecular aggregates have an average particle size between about 5 μm to about 20 μm, wherein the process comprises
a) preparing a pharmaceutical composition comprising 100 IU-1000 IU of Insulin Glargine, wherein the pH of the composition is 3.0-4.5.
b) preparing a pharmaceutical composition comprising 100 IU-1000 IU of one or more rapid, short or intermediate acting insulin, wherein the pH of the composition is 6.0-8.0.
c) adding the insulin glargine composition of step a) in a vial
d) adding the composition of rapid, short or intermediate acting insulin of step b) in the vial of step c) containing insulin glargine composition of step a) such that the final pH of the composition is pH 6.5-8.0.

In one aspect of the present invention, there is provided a process for preparing a pharmaceutical composition comprising readily dissociable molecular aggregates formed by combining an insulin, insulin analogue, derivative or metabolite having isoelectric point between 5.8 to 8.5 with one or more an insulin, insulin analogue, derivative or metabolite having isoelectric point between 4.0 to 5.7 optionally along with one or more excipients, wherein the said molecular aggregates have an average particle size between about 5 μm to about 20 μm, wherein the process comprises
a) preparing a pharmaceutical composition comprising 100 IU-1000 IU of Insulin Glargine, wherein the pH of the composition is 3.0-4.5.
b) preparing a pharmaceutical composition comprising 100 IU-1000 IU of one or more rapid, short or intermediate acting insulin, wherein the pH of the composition is 6.0-8.0.
c) mixing the insulin glargine composition of step a) with the composition of rapid, short or intermediate acting insulin of step b) such that the final pH of the composition is pH 6.5-8.0 and the total volume of the composition is 1 ml.

The example given below serves to illustrate embodiments of the present invention. However it does not intend to limit the scope of present invention.

### Examples 1

### Pharmaceutical Composition

A. Preparation of Solution of Glargine

TABLE 1
Composition of Insulin Glargine Solution

| Sr. No. | Ingredients | Quantity/mL | |
|---|---|---|---|
| 1 | Insulin Glargine (r-DNA) | 100 | IU |
| 2 | Metacresol | 1.5-4.5 | |
| 3 | Glycerol, 85% | 10-30 | |
| 4 | Zinc as Zinc Chloride* | 0.01-0.06 | |
| 5 | Sodium Hydroxide | Q.S. to pH | |
| 6 | Hydrochloric Acid | Q.S. to pH | |
| 7 | Water for Injection | Q.S. to 1.0 | mL |

*The amount of zinc chloride added to the formulation is dependant on the amount of zinc in the Insulin Glargine used. The target amount for zinc in the formulation is 30.0 μg per 100 IU Insulin Glargine.

Procedure:

The pharmaceutical composition described in Table 1 was prepared according to the procedure detailed below:

Step 1: Preparation of API Solution

Zinc-containing crystals of Insulin Glargine were dissolved in water for injection with the help of few μL of 1M HCl. The endogenous zinc level was supplemented by adding appropriate volume of zinc chloride solution (1% w/v).

Step 2: Preparation of Preservative/Stabilizer Solution

Preservative/Stabilizer solution was prepared by dissolving metacresol, glycerol in water for injection to get final concentration as 25 mM m-Cresol, 217 mM Glycerol 85%.

Step 3: Preparation of Final Solution

Both API solution of Step 1 and preservative/stabilizer solution of Step 2 were diluted to final concentrations after mixing and pH was adjusted to 4.0±0.1 with 1M HCl or 1M NaOH. The solution was then filtered with 0.2 micron filter in a sterile container.

B. Preparation of Solution of Insulin Aspart

TABLE 2
Composition of Insulin Aspart Solution

| Sr. No. | Ingredients | Quantity/mL | |
|---|---|---|---|
| 1 | Insulin Aspart (r-DNA) | 100 | IU |
| 2 | Metacresol | 1.5 | mg |
| 3 | Phenol | 1.72 | mg |
| 4 | Glycerol | 16.00 | mg |
| 5 | Disodium hydrogen phosphate Dihydrate | 1.25 | mg |
| 6 | Sodium Hydroxide | Q.S. to pH | |
| 7 | Hydrochloric Acid | Q.S. to pH | |
| 8 | Water for Injection | Q.S. to 1.0 | mL |

Procedure:

The pharmaceutical composition described in Table 2 was prepared according to the procedure detailed below:

Step 1: Preparation of API Solution

A solution of Insulin Aspart was prepared by dissolving Insulin Aspart in water for injection with the help of few μL of 1M HCl.

Step 2: Preparation of Buffer Solution

Buffer solution was prepared by dissolving metacresol, phenol, disodium hydrogen phosphate and glycerol in water for injection to get final concentration as as 14 mM m-Cresol, 18 mM Phenol, 7 mM Disodium Hydrogen phosphate and 174 mM Glycerol.

Step 3: Preparation of Final Solution

Both API Solution of Step 1 and Buffer Solution of Step 2 were Diluted to Final concentrations after mixing and pH was adjusted to 7.2±0.2 with 1M HCl or 1M NaOH. The solution was then filtered with 0.2 micron filter in a sterile container.

C. Mixture of Glargine and Aspart

The solution of Insulin Aspart was added slowly to the Insulin Glargine solution so as to obtain the ratio of 20:80 (Insulin Aspart: Insulin Glargine). The suspension was mixed thoroughly. The pH of the suspension was 7.2. This preparation was introduced into vials and subjected to stability testing.

### Example 2

### Insulin Glargine Crystal Images

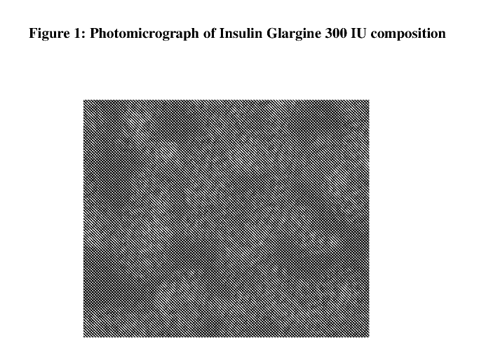

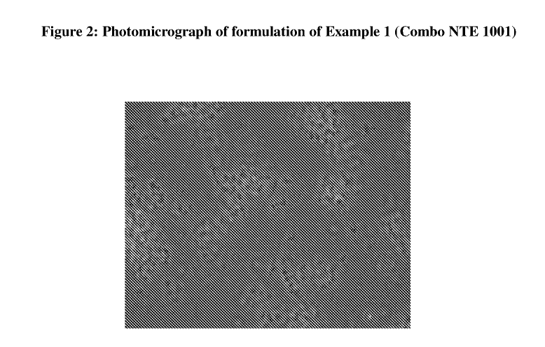

The formulations of Example 1 (Combo NTE 1001) and Insulin Glargine composition 300 IU/mL and were observed under the optical microscope (Olympus BX40). The sample was prepared by the following way:
1. The pH of the Insulin Glargine 300 IU/mL sample was raised to 7.2 by the addition of phosphate buffer of pH 9.5. The sample was taken on glass slide and observed under 100× oil immersion microscope (Olympus BX40). FIG. 1 shows the photomicrograph of Insulin glargine 300 IU/ml.
2. The photomicrograph of the formulation of Example 1 (Combo NTE 1001) having pH 7.2 was also taken and represented in FIG. 2.

1. A pharmaceutical composition comprising readily dissociable molecular aggregates formed by combining (i) an insulin, insulin analogue, derivative or metabolite having isoelectric point between 5.8 to 8.5, and (ii) one or more of insulin, insulin analogue, derivative or metabolite having isoelectric point between 4.0 to 5.7, optionally along with one or more excipients, wherein the said molecular aggregates have an average particle size between about 5 μm to about 20 μm.

2. The pharmaceutical composition of claim 1, wherein the pH of the composition is adjusted to a pH of between 6.0 and 8.5.

3. The pharmaceutical composition of claim 1, wherein the insulin, insulin analogue, derivative or metabolite is present in concentration range of 100 IU-1000 IU/ml.

4. The pharmaceutical composition according to claim 1, wherein the insulin, insulin analogue, derivative or metabolite having isoelectric point between 5.8 to 8.5 is insulin glargine.

5. The pharmaceutical composition of claim 1, wherein one or more of insulin analogue or derivative having isoelectric point between 4.0 to 5.7 are selected from the group consisting of recombinant human insulin, Insulin NPH, Insulin Lispro, Insulin Lispro Protamine, Insulin Glulisine, Insulin Aspart, Insulin Aspart Protamine, A21Gly Insulin, A21Gly B28Lys insulin, A21Gly B28Lys B29Pro Insulin and A21Gly B28Asp Insulin.

6. The pharmaceutical composition according to claim 5, wherein one or more of insulin, insulin analogue, derivative or metabolite having isoelectric point between 4.0 to 5.7 is Insulin Aspart or A21Gly insulin.

7. The pharmaceutical composition according to claim 1, wherein the composition is devoid of dextran polymer.

8. The pharmaceutical composition according to claim 1, wherein the excipients are selected from the group consisting of isotonic agent, surfactant, buffer, zinc or salt thereof, preservatives, pH modifying agents, stabilizing agents and solubilizing agents.

9. The pharmaceutical composition according to claim 1, wherein (i) the insulin, insulin analogue, derivative or metabolite having isoelectric point between 5.8 to 8.5 is Insulin glargine, and (ii) the insulin, insulin analogue, derivative or metabolite having isoelectric point between 4.0 to 5.7 is Insulin Aspart or A21Gly insulin.

10. The pharmaceutical composition according to claim 9, wherein the composition comprises 100 IU-300 IU/ml of Insulin glargine and 100 IU 300 IU/ml of Insulin aspart or A21Gly insulin.

11. The pharmaceutical composition according to claim 9, wherein the composition comprises 100 IU/ml of Insulin glargine and 100 IU/ml of Insulin aspart or A21Gly insulin.

12. The process for preparing a pharmaceutical composition according to claim 1, wherein said process comprise steps of:
a. preparing a pharmaceutical composition comprising 100 IU-1000 IU of insulin analogue or derivative having isoelectric point between 5.8 to 8.5, wherein the pH of the composition is 3.0-4.5.
b. preparing a pharmaceutical composition comprising 100 IU-1000 IU of one or more insulin analogue or derivative having isoelectric point between 4.0 to 5.7, wherein the pH of the composition is 6.0-8.0.
c. adding the composition of step b in the composition of step a and adjusting the final pH of the composition to pH 6.5-8.0.

13. The process of claim 12, wherein one or more insulin analogue or derivative having isoelectric point between 4.0 to 5.7 are selected from the group consisting of recombinant human insulin, Insulin NPH, Insulin Lispro, Insulin Lispro Protamine, Insulin Glulisine, Insulin Aspart, Insulin Aspart Protamine, A21Gly Insulin, A21Gly B28Lys Insulin, A21Gly B28Lys B29Pro Insulin and A21Gly B28Asp Insulin.

14. The process of claim 13, wherein one or more an insulin, insulin analogue, derivative or metabolite having isoelectric point between 4.0 to 5.7 is Insulin Aspart or A21Gly insulin.

15. The process of claim 12, wherein the insulin, insulin analogue, derivative or metabolite having isoelectric point between 5.8 to 8.5 is insulin glargine.

16. The process of claim 12, wherein one or more excipients selected from the group consisting of isotonic agent, surfactant, buffer, zinc or salt thereof, preservatives, pH modifying agents, stabilizing agents and solubilizing agents.

17. A method for treating Type I and Type II Diabetes Mellitus in a patient comprising administering to said patient a pharmaceutical composition comprising readily dissociable molecular aggregates formed by combining (i) an insulin, insulin analogue, derivative or metabolite having isoelectric point between 5.8 to 8.5, and (ii) one or more of insulin, insulin analogue, derivative or metabolite having isoelectric point between 4.0 to 5.7, optionally along with one or more excipients, wherein the said molecular aggregates have an average particle size between about 5 μm to about 20 μm.

18. The method of extending the duration of exposure of a long acting insulin in the treatment of Type I and Type II Diabetes Mellitus in a patient comprising administering to said patient in need thereof the pharmaceutical composition of claim 1.

19. The method of reducing the nocturnal hypoglycemia, wherein nocturnal hypoglycemia in patients suffering from Type I or Type II diabetes, wherein said method comprise administering to the said patient in need thereof the pharmaceutical composition of claim 1.
